(19) United States
(12) Patent Application Publication
(10) Pub. No.: US 2012/0150093 A1
(43) Pub. Date: Jun. 14, 2012

(54) SYNOVIAL SHUNTS

(75) Inventors: Nir Altschuler, Hod Hasharon (IL); Dvir Keren, Tel Aviv (IL); Amir Goren, Yehud (IL); Roey Shafrir, Modi'in (IL)

(73) Assignee: JOINTECH MED. LTD., Ariel (IL)

(21) Appl. No.: 13/262,239

(22) PCT Filed: Apr. 6, 2010
(86) PCT No.: PCT/IL10/00265

§ 371 Date: Mar. 2, 2012

Related U.S. Application Data
(60) Provisional application No. 61/164,580, filed on Mar. 30, 2009.

(51) Int. Cl. A61M 5/00

(52) U.S. Cl. 604/8

(57) ABSTRACT

Shunt for joint repair, creates a passageway between interior and exterior of a joint capsule, the shunt comprising a body of biocompatible material, which is substantially hollow; a proximal and distal aperture flanking said shunt body; at least a first and second extension of shunt body, first extension is located proximal to proximal aperture and second extension is located proximal to distal aperture, first and second extensions fasten or adjoin said shunt body, such that said shunt is substantially immobilized at a location of placement of said shunt; proximal aperture of shunt body is positioned proximal to an interior of joint capsule and shunt body spans at least a distance equal to that of synovial membrane, joint capsule or a combination thereof and distal aperture is located substantially outside of at least synovial membrane; the ratio between shunt body diameter and shunt body length is greater than 0.5.

### BACKGROUND OF THE INVENTION

Joint disease is often associated with or aggravated by inflammation localized to the affected joints and is often marked by intense joint pain and ultimate joint destruction.

Diarthrodial or synovial joints allow movement and transfer of load between bones. Disease or injury of these joints, in turn, has a major impact in man and in animals.

The highly mobile diarthrodial joints of the body have similar structures and components including: the joint capsule, or outer membrane, which encases the joint; collateral ligaments which are intra-capsular and provide support and stability for the joint (these work in conjunction with supporting muscle, other extra-capsular ligaments, tendons and connective tissue); articular cartilage which covers the ends of the articulating bones within the joint; subchondral bone, which provides structural support to the overlying articular cartilage; the synovium, a modified mesenchyme; and synovial fluid which lubricates and nourishes the joint surfaces.

The joint capsule consists of a thick fibrous portion, which is lined by a thinner subsynovium (lamina propria) and the synovium (synovial membrane). The synovium or inner lining of the joint capsule consists of cells, synoviocytes, which have both secretory and phagocytic functions. Synovial lining cells synthesize hyaluronan (hyaluronic acid or HA) that is secreted into the synovial fluid, which occupies the intra-articular space.

Under normal conditions, the body maintains the synovial joint in state of homeostasis through a variety of complex hormonal and mechanical feedback mechanisms. Two types of insult or injury can upset the delicate homeostatic balance. Repeated trauma or stress (slow chronic insult) to the joint during everyday use, is often the inciting cause of joint inflammation and loss of homeostasis. Initially, such stress results in only soft tissue inflammation in the form of synovitis or capsulitis (e.g., traumatic synovitis). Cartilage damage may or may not initially be present in the early stages of stress related injury or inflammation. However, the release of inflammatory mediators into the joint such asprostaglandins, cytokines, lysosomal enzymes and free radicals can lead to damage of articular cartilage and can cause cartilage degradation and can lead to development of degenerative joint disease (DJD).

A second type of insult or injury, the osteochondral defect, e.g., a chip fracture, is often associated with an acute mechanical failure or traumatic injury, although, such a fracture can be due to secondary complications associated with chronic DJD. Under this scenario, the lesion often starts as a traumatically induced defect in the articular cartilage. This may occur as a fragmentation of the original tissue from the joint margins or other defect which compromises the surface and integrity of the articular cartilage. Exposure of the supporting subchondral bone to synovial fluid and the intermittent pressures of the synovial fluid generated by repeated joint movement (repeated stress andtrauma of training or racing) can lead to progressive subchondral bone sclerosis and eventual dislodging of the chip or bone fragment. Left untreated, the resulting damage often becomes progressive.

There have been countless therapeutic approaches for management of joint disease. Chief among these is the nutritional supplementation of metabolic precursors to the diet to aid in the biosynthesis of proteoglycans, GAG's, hyaluronan, and collagen, use of anti-inflammatory treatments, periodic drainage of accumulated fluid and in more severe instances, joint replacement.

All of these treatments unfortunately cause severe side effects and are not particularly effective. Thus an ideal treatment or procedure for treating joint inflammation is as yet lacking.

### SUMMARY OF THE INVENTION

In one embodiment, the present invention provides a synovial shunt for insertion in a body joint, which shunt creates a passageway between an interior of a joint capsule and an exterior of said joint capsule, said shunt comprising:
a shunt body comprised of a biocompatible material, which is substantially hollow;
a proximal and distal aperture flanking said shunt body; and
at least a first and second extension of said shunt body, which first extension is located proximal to said proximal aperture and which second extension is located proximal to said distal aperture, wherein said first and second extensions fasten or adjoin said shunt body, such that said shunt is substantially immobilized at a location of placement of said shunt;
   wherein
   said proximal aperture of said shunt body is positioned proximal to an interior of said joint capsule and said shunt body spans at least a distance substantially equal to that of said synovial membrane, said joint capsule or a combination thereof and said distal aperture is located substantially outside of at least said synovial membrane; and
   the ratio between said shunt body diameter and said shunt body length is greater than 0.5.

This invention provides a therapeutic kit comprising the synovial shunts of this invention. In some embodiments, the kits of this invention further comprise a biocompatible tubing, which tubing is positioned proximal to said distal aperture and which tubing conveys synovial fluid away from an affected joint region. In some embodiments, the kits of this invention further comprise a tool for the insertion of said synovial shunt in an affected joint region. In some embodiments, the tool is comprised of at least two parts, said first part comprising a pointed structure, which inserts within a joint capsule and said second part, operationally connected thereto, which second part delivers said synovial shunt to said joint capsule such that a proximal aperture of said shunt body is positioned proximal to an interior of said joint capsule. In some embodiments, the second part maintains said first extension and said second extension at a position that is substantially parallel to a long axis of said shunt body prior to positioning said shunt in an affected joint. In some embodiments, the operation of said first and second part to insert said shunt into an affected joint region facilitates extension of said first extension and said second extension to be at an angle of between 45 to 120 degrees with respect to a long axis of said shunt body.

In some embodiments, this invention provides a method for draining synovial fluid from a joint in a subject in need thereof, the method comprising the step of affixing the synovial shunt of claim 1 in a synovial membrane in an affected joint such that said proximal aperture is positioned proximal to an interior of said joint capsule and said shunt body spans at least a distance substantially equal to that of said synovial membrane, said joint capsule or a combination thereof and said distal aperture is located substantially outside at least said synovial membrane, creating a passageway through said synovial membrane and providing an exit for excess synovial fluid in said joint, thereby being a method for draining synovial fluid from a joint in a subject.

In some embodiments, this invention provides a method for treating a joint disease or disorder in a subject, the method comprising the step of affixing a synovial shunt of this invention in a synovial membrane in an affected joint of said subject, such that said proximal aperture is positioned proximal to an interior of said joint capsule and said shunt body spans at least a distance substantially equal to that of said synovial membrane, said joint capsule or a combination thereof and said distal aperture is located substantially outside at least said synovial membrane, creating a passageway through said synovial membrane and providing an exit for synovial fluid, relief of intra joint pressure or a combination thereof in said joint, thereby being a method for treating a joint disease or disorder in a subject.

In some embodiments, this invention provides a method for treating a ganglion cyst in a subject, the method comprising the step of affixing a synovial shunt of this invention in a membrane of a ganglion cyst in a subject, such that said proximal aperture is positioned proximal to an interior of said cyst and said shunt body spans at least a distance substantially equal to that of said cyst membrane and said distal aperture is located substantially outside at least said cyst membrane, creating a passageway through said cyst membrane and providing an exit for fluid from within said ganglion cyst, thereby being a method for treating a ganglion cyst in a subject.

### BRIEF DESCRIPTION OF THE DRAWINGS

FIG. 1 is a photograph of an embodied shunt of this invention at an early stage of shunt preparation.

FIG. 2 shows positioning of an embodiment of a shunt of this invention on a tool, for further processing of the shunt extensions.

FIGS. 3A and 3B shows positioning of an embodiment of a shunt of this invention on different tools, for further processing of the shunt extensions to achieve desired positioning of the extensions.

FIG. 4 is a photograph of an embodied shunt of this invention, containing extensions positioned at an angle of roughly 90 degrees, with respect to the long axis of the shunt body.

FIG. 5A-5C schematically depicts an embodiment of a tool of this invention, for insertion of a shunt in a repair site.

FIG. 6 depicts alternate views of an embodiment of a tool of this invention, showing tool dimensions and highlighting certain elements of the tool and operation of the tool. FIG. 6A depicts dimensions of certain embodiments of tools of the invention. FIG. 6B highlights certain features of embodied tools of the invention. FIGS. 6C 1-5 depicts another embodiment of a tool of this invention, highlighting the dimensions and key elements of the embodied tool. FIG. 6D-G highlights deployment of the shunt via an embodied tool of this invention. FIG. 6D depicts the tool containing the shunt in a locked position within the overtube on the dilator. FIG. 6E demonstrates that after a single press on the trigger stopper, the dilator is terminally exposed allowing for deployment of the proximal extension. FIGS. 6F and 6G demonstrate depressing the trigger stopper a second time, revealing the distal extension and facilitating its controlled deployment, such that full deployment of both extension occurs.

FIG. 7A-7E depicts embodied shunts of this invention. FIGS. 7A-7B depict an embodied shunt containing extensions positioned at an angle of roughly 45 degrees, with respect to the long axis of the shunt body. FIGS. 7C-D depict another embodiment of a shunt having a screw-like lateral extension, and showing embodied dimensions of such shunts. FIG. 7E depicts other embodied shunt, showing varied proximal and distal extensions within the same embodied shunt.

FIG. 8 depicts embodiments of positioning of the shunts of this invention within a knee joint. FIG. 8A demonstrates positioning of an embodied shunt within a goat knee joint. FIG. 8B schematically depicts other potential sites of positioning of embodied shunts of this invention and the relationship of such positioned shunt to other elements of the joint region.

### DETAILED DESCRIPTION OF THE PRESENT INVENTION

This invention provides, inter alia, shunts, tools and methods of use thereof for, inter alia treating joints in a subject. This invention further provides kits for treatment of joint tissue in a subject.

In one embodiment, the present invention provides a synovial shunt for insertion in a body joint, which shunt creates a passageway between an interior of a joint capsule and an exterior of said joint capsule, said shunt comprising:
a shunt body comprised of a biocompatible material, which is substantially hollow;
a proximal and distal aperture flanking said shunt body; and
at least a first and second extension of said shunt body, which first extension is located proximal to said proximal aperture and which second extension is located proximal to said distal aperture, wherein said first and second extensions fasten or adjoin said shunt body, such that said shunt is substantially immobilized at a location of placement of said shunt;
   wherein
   said proximal aperture of said shunt body is positioned proximal to an interior of said joint capsule and said shunt body spans at least a distance substantially equal to that of said synovial membrane, said joint capsule or a combination thereof and said distal aperture is located substantially outside of at least said synovial membrane; and
   the ratio between said shunt body diameter and said shunt body length is greater than 0.5.

The invention provides a shunt, which in one embodiment, refers to a biocompatible tube, which tube is relatively non-elastic and can be fixed at a point of insertion via the presence of extensions from the tube at both termini of the tube, which extensions abut, adhere or are otherwise affixed at desired locations and wherein the tube spans at least from an interior region of a joint capsule across the synovial membrane, or in some embodiments, spans the joint capsule or in some embodiments, spans the joint capsule and terminates in a subcutaneous tissue. The tube therefore creates a passageway or access between the inside of a joint capsule and at least a region immediately external to the synovial membrane of the affected joint.

The shunt is a tube defined by its terminally located apertures, which are positioned proximally and distally, respectively, to a synovial membrane of an affected joint.

In one embodiment, the term “proximal” refers to something being situated close to a particular locale. In one embodiment, the term “distal” refers to the indicated article being situated far from a particular locale. The terms “proximal” and “distal” are to be understood to be relative terms, in that their use designates a positioning relative to a particular viewpoint. For example, the positioning of the proximal aperture within the internal face of the synovial membrane indicates the synovial membrane is the reference vantage point, and the distal aperture, therefore, relative to the proximal aperture, is situated away from the internal face of the synovial membrane.

In one embodiment, the distal aperture is located proximal to a subcutaneous tissue and said shunt body spans a length across a synovial membrane and extends into a subcutaneous tissue, which in one embodiment is a muscle, a vein, fat, a ligament or a tendon and in another embodiment, said distal aperture is located near an exterior of a joint capsule.

In some embodiments, the shunt body is substantially hollow, and in some embodiments, the internal diameter of the shunt approximates the external diameter of the shunt and in some embodiments, the internal diameter of the shunt is smaller than the external diameter of the shunt and in some embodiments, the internal diameter of the shunt is substantially smaller than the external diameter of the shunt. In some embodiments, such differences in terms of the internal circumference versus external circumference of the shunt body may be referred to as a thickness of a shunt body wall.

The shunt interior does not comprise a valve.

In one embodiment, the shunt body has a wall thickness of between about 50-500 μm. In some embodiments, the shunt body has a wall thickness of between about 150-275 μm or in some embodiments, the shunt body has a wall thickness of between about 225-300 μm, or in some embodiments, the shunt body has a wall thickness of between about 250-500 μm.

In one embodiment of this invention, “about” refers to a quality wherein the means to satisfy a specific need is met, e.g., the size may be largely but not wholly that which is specified but it meets the specific need, e.g. the need of repair at a site of joint repair. In one embodiment, “about” refers to being closely or approximate to, but not exactly. A small margin of error is present. This margin of error would not exceed plus or minus the same integer value.

In some embodiments, the margin of error is within 1%, or in some embodiments, the margin of error is within 2%, or in some embodiments, the margin of error is within 5%, or in some embodiments, the margin of error is within 10%.

In one embodiment, the ratio between the internal shunt body diameter and shunt body length is greater than 0.5, and in some embodiments, the ratio between the internal shunt body diameter and shunt body length is between 0.5 and 5, or in some embodiments, the ratio between the internal shunt body diameter and shunt body length is between 0.1 and 5, or in some embodiments the ratio between the internal shunt body diameter and shunt body length is between 0.1 and 10, or in some embodiments, the ratio between the internal shunt body diameter and shunt body length is between 0.1 and 50.

In one embodiment of this invention, the shunts of this invention comprise an essentially exposed device. The term “exposed” refers to being open to the surrounding environment such that contact may occur between a shunt of this invention and the surrounding environment. In one embodiment, the term “exposed” refers to the availability of a shunt surface for interaction with agents promoting joint treatment and/or repair. In one embodiment, the shunt may comprise a therapeutic coating, wherein the coating is accessible/open/available to a site of tissue treatment and/or repair. In one embodiment, an exposed surface of this invention has access to effector compounds beneficial for tissue treatment and/or repair.

In one embodiment of this invention, the phrases “long axis” refers to a line extending parallel to the shunt lengthwise. The term “lengthwise” refers the direction of the length of a shunt of this invention.

In one embodiment, the shunt body has an internal diameter of between about 1-25 mm and in another embodiment, the shunt body has an internal diameter of between about 5-10 mm. In another embodiment, the shunt body has a length of between about 3-10 mm.

In one embodiment, the shunt body length varies as a function of the thickness of subcutaneous tissue into which the synovial shunt will be implanted, as will be appreciated by the skilled artisan.

In another embodiment, the shunt is comprised of a metal or a metal alloy, a ceramic or a polymer and in one embodiment, the shunt is prepared from a single piece of metal.

In one embodiment, the metal is nitinol, stainless steel or titanium.

In one embodiment, the polymer comprises a natural polymer comprising, collagen, elastin, silk, hyaluronic acid, chytosan, and any combinations thereof and in one embodiment, the polymer comprises a synthetic biodegradable polymer.

In one embodiment, the coating of the shunts of this invention may have a thickness of between about 2.0 nm and 0.1 μm. In some embodiments, the coating of the shunts of this invention may have a thickness of between about 2.0 nm and 0.5 μm. In some embodiments, the coating of the shunts of this invention may have a thickness of between about 1.0 nm and 1 μm.

In one embodiment, the synthetic biodegradable polymer comprises alpha-hydroxy acids including poly-lactic acid, polyglycolic acid, enantioners thereof, co-polymers thereof, polyorthoesters, and combinations thereof.

In one embodiment of this invention, the shunt comprises a coating. In one embodiment, the coating is a polymer coating and comprises a natural polymer comprising, collagen, elastin, silk, hyaluronic acid, chytosan, and any combinations thereof.

In one embodiment, the shunt and/or shunt coating comprises a polymer comprising synthetically modified natural polymers, and may include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt.

In one embodiment, of this invention, the polymer comprises a synthetic biodegradable polymer. In one embodiment of this invention, a synthetic biodegradable polymer comprises alpha-hydroxy acids including poly-lactic acid, polyglycolic acid, enantioners thereof, co-polymers thereof, polyorthoesters, and combinations thereof.

In one embodiment, the polymer comprises a poly(cianoacrylate), poly(alkyl-cianoacrylate), poly(ketal), poly(caprolactone), poly(acetal), poly(α-hydroxy-ester), poly(α-hydroxy-ester), poly(hydroxyl-alkanoate), poly(propylene-fumarate), poly(imino-carbonate), poly(ester), poly(ethers), poly(carbonates), poly(amide), poly(siloxane), poly(silane), poly(sulfide), poly(imides), poly(urea), poly(amide-enamine), poly(organic acid), poly(electrolytes), polyp-dioxanone), poly(olefin), poloxamer, inorganic or organomatallic polymers, elastomer, or any of their derivatives, or a copolymer obtained by a combination thereof.

In one embodiment, the polymer comprises poly(D,L-lactide-co-glycolide) (PLGA). In another embodiment, the polymer comprises poly(D,L-lactide) (PLA). In another embodiment, the polymer comprises poly(D,L-glycolide) (PGA). In one embodiment, the polymer comprises a glycosaminoglycan.

In one embodiment, the polymer comprises synthetic degradable polymers, which may include, but are not limited to polyhydroxy acids, such as poly(lactide)s, poly(glycolide)s and copolymers thereof; poly(ethylene terephthalate); poly(hydroxybutyric acid); poly(hydroxyvaleric acid); poly[lactide-co-(ε-caprolactone)]; poly[glycolide-co(ε-caprolactone)]; poly(carbonate)s, poly(pseudo amino acids); poly(amino acids); poly(hydroxyalkanoate)s; poly(anhydrides); poly(ortho esters; and blends and copolymers thereof.

In one embodiment of this invention, the polymer comprises proteins such as zein, modified zein, casein, gelatin, gluten, serum albumin, collagen, actin, α-fetoprotein, globulin, macroglobulin, cohesin, laminin, fibronectin, fibrinogen, osteocalcin, osteopontin, osteoprotegerin, or others, as will be appreciated by one skilled in the art. In another embodiment, a polymer may comprise cyclic sugars, cyclodextrins, synthetic derivatives of cyclodextrins, glycolipids, glycosaminoglycans, oligosaccharide, polysaccharides such as alginate, carrageenan (χ, λ, μ, κ), chitosane, celluloses, condroitin sulfate, curdlan, dextrans, elsinan, furcellran, galactomannan, gellan, glycogen, arabic gum, hemicellulose, inulin, karaya gum, levan, pectin, pollulan, pullulane, prophyran, scleroglucan, starch, tragacanth gum, welan, xanthan, xylan, xyloglucan, hyaluronic acid, chitin, or a poly(3-hydroxyalkanoate)s, such as poly(β-hydroxybutyrate), poly(3-hydroxyoctanoate) or poly(3-hydroxyfatty acids), or any combination thereof.

In one embodiment, the polymer comprises a bioerodible polymer such as poly(lactide-co-glycolide)s, poly(anhydride)s, and poly(orthoester)s, which have carboxylic groups exposed on the external surface as the smooth surface of the polymer erodes, which may also be used. In one embodiment, the polymer contains labile bonds, such as polyanhydrides and polyesters.

In one embodiment, a polymer may comprise chemical derivatives thereof (substitutions, additions, and elimination of chemical groups, for example, alkyl, alkylene, hydroxylations, oxidations, and other modifications routinely made by those skilled in the art), blends of, e.g. proteins or carbohydrates alone or in combination with synthetic polymers.

In one embodiment of this invention, the polymer is biodegradable. In one embodiment, the term “biodegradable” or grammatical forms thereof, refers to a material of this invention, which is degraded in the biological environment of the subject in which it is found. In one embodiment, the biodegradable material undergoes degradation, during which, acidic products, or in another embodiment, basic products are released. In one embodiment, bio-degradation involves the degradation of a material into its component subunits, via, for example, digestion, by a biochemical process. In one embodiment, biodegradation may involve cleavage of bonds (whether covalent or otherwise), for example in a polymer backbone of this invention. In another embodiment, biodegradation may involve cleavage of a bond (whether covalent or otherwise) internal to a side-chain or one that connects a side chain to, for example a polymer backbone.

In one embodiment, a shunt of this invention is covalently associated with the polymer coating via the use of a cross-linking agent. In one embodiment, the phrase “cross-linking agent” refers to an agent, which facilitates the formation of a covalent bond between 2 atoms. In one embodiment, the cross-linking agent is a zero-length cross-linking agent.

In one embodiment, the cross-linking agent is (1 ethyl 3-(3-dimethyl aminopropyl)carbodiimide (EDAC), N-Sulfohydroxy succinamide (Sulfo NHS), 5-iodopyrimidines, N-carbalkoxydihydroquinolines, pyrroloquinolinequinones, or a combination thereof.

In one embodiment, the cross-linking agent is a homobifunctional cross-linker, such as, for example, a N-hydroxysuccinimide ester (e.g. disuccinimidyl suberate or dithiobis(succinimidylpropionate), homobifunctional imidoester (e.g. dimethyladipimidate or dimethyl pimelimidate), sulfhydryl-reactive crosslinker (e.g. 1,4-di-[3′-(2′-pyridyldithio)propionamido]butane), difluorobenzene derivative (e.g. 1,5-difluoro-2,4-dinitrobenzene), aldehyde (e.g. formaldehyde, glutaraldehyde), bis-epoxide (e.g. 1,4-butanediol diglycidyl ether), hydrazide (e.g. adipic acid dihydrazide), bis-diazonium derivative (e.g. o-tolidine), bis-alkylhalide, or a combination thereof.

In one embodiment, the cross-linking agent is a hetero-bifunctional cross-linker, such as, for example, an amine-reactive and sulfhydryl-reactive crosslinker (e.g. N-succinimidyl 3-(2-pyridyldithio)propionate, a carbonyl-reactive and sulfhydryl-reactive crosslinker (e.g. 4-(4-N-maleimidophenyl)butyric acid hydrazide), or a combination thereof.

In some embodiments, the cross-linking agent is a trifunctional cross-linkers, such as, for example, 4-azido-2-nitrophenylbiocytin-4-nitrophenyl ester, sulfosuccinimidyl-2-[6-biotinamido]-2-(p-azidobenzamido)hexanoamidolethyl-1,3′-dithiopropionate (sulfo-SBED), or a combination thereof.

In another embodiment, the cross-linking agent is an enzyme. In one embodiment of this invention, the cross-linking agent comprises a transglutaminase, a peroxidase, a xanthine oxidase, a polymerase, or a ligase, or a combination thereof.

The choice of concentration of the cross-linking agent utilized for activity will vary, as a function of the volume, agent and polymer chosen, in a given application, as will be appreciated by one skilled in the art.

In some embodiments, such coatings comprising polymers are positioned on an external surface of the shunts of this invention, to promote fixation or containment of the shunt within the subcutaneous tissue, and in one embodiment, the internal surface of the shunt comprises no such coating. In some embodiments, the coating on an internal surface and an external surface of the shunt will differ.

In one embodiment, a shunt of this invention incorporates or comprises an effector compound. In one embodiment, the effector compound comprises a component of a kit of this invention for use for incorporation into a shunt of this invention as herein described.

In one embodiment of this invention, the effector compound comprises a cytokine, a growth factor, a bone morphogenetic protein (BMP), a therapeutic compound, an anti-inflammatory compound or an antibiotic, or any combination thereof.

In one embodiment of this invention, the phrase “a therapeutic compound” refers to a peptide, a protein or a nucleic acid, or a combination thereof. In another embodiment, the therapeutic compound is an antibacterial, antiviral, antifungal or anti-parasitic compound. In another embodiment, the therapeutic compound has cytotoxic or anti-cancer activity. In another embodiment, the therapeutic compound is an enzyme, a receptor, a channel protein, a hormone, a cytokine or a growth factor. In another embodiment, the therapeutic compound is immuno-stimulatory. In another embodiment, the therapeutic compound inhibits inflammatory or immune responses. In one embodiment, the therapeutic compound comprises a pro-angiogenic factor. In some embodiments, the therapeutic compound is an anti-inflammatory compound.

In any of the embodiments herein, the therapeutic compound may comprise compounds such as, for example, antioxidants, growth factors, cytokines, antibiotics, anti-inflammatories, immunosuppressors, preservative, pain medication, other therapeutics, and excipient agents. In one embodiment, examples of growth factors that may be administered include, but are not limited to, epidermal growth factor (EGF), transforming growth factor-alpha (TGF-β), transforming growth factor-beta (TGF-β), human endothelial cell growth factor (ECGF), granulocyte macrophage colony stimulating factor (GM-CSF), bone morphogenetic protein (BMP), nerve growth factor (NGF), vascular endothelial growth factor (VEGF), fibroblast growth factor (FGF), insulin-like growth factor (IGF), cartilage derived morphogenetic protein (CDMP), platelet derived growth factor (PDGF), or any combinations thereof.

In one embodiment, the phrase “a therapeutic compound”, refers to a molecule, which when provided to a subject in need, provides a beneficial effect. In some cases, the molecule is therapeutic in that it functions to replace an absence or diminished presence of such a molecule in a subject. In other embodiments, the molecule stimulates a signaling cascade that provides for expression, or secretion, or others of a critical element for cellular or host functioning.

In one embodiment, the effector compound comprises, an immunomodulatory, an anticoagulant, an antibody, a growth factor, a hormone, a DNA, an siRNA, or a vector or any combination thereof.

In one embodiment, the phrase “effector compound” refers to any agent or compound, which has a specific purpose or application which is useful in the treatment, prevention, inhibition, suppression, delay or reduction of incidence of a disease, a disorder, or a condition, when applied to the shunts, kits and/or methods of this invention.

In one embodiment of this invention, term “effector compound” is to be understood to include the terms “drug” and “agent”, as well, when referred to herein, and represents a molecule whose incorporation within the shunt and/or kits of this invention, or whose use thereof, is desired.

In some embodiments, multiple shunts are implanted into a repair site. In some embodiments, shunts may be positioned at different regions within a joint capsule, and in some embodiments, multiple shunts are positioned within more than one joint capsule within a subject. In some embodiments, multiple shunts may be implanted in two comparable joints (for example within both knees of a subject), or in some embodiments, multiple shunts may be implanted within different joints of the body of a subject.

In another embodiment, a luminal surface of said shunt, an exterior surface of said shunt or a combination thereof is treated to reduce adhesion of cells or particulate matter thereto. In another embodiment, the shunt comprises a coating, which diminishes or abrogates adhesion thereto. In another embodiment the shunt comprises a positively charged material or incorporates a positively charged material. In one embodiment, the first extension, said second extension or a combination thereof are treated to reduce adhesion of cells or particulate matter thereto. In one embodiment, the first extension, said second extension or a combination thereof are treated to promote adhesion to cells in a region to which said shunt is adhered. In one embodiment, a luminal surface of said shunt, an exterior surface of said shunt or a combination thereof comprises a therapeutic agent.

In some embodiments, the therapeutic agent comprises a growth factor, an agent, which aides in wound repair, or a combination thereof. In some embodiments, the therapeutic agent comprises an anticoagulant, an anti-inflammatory compound, or a combination thereof.

In some embodiments, the first extension, said second extension or a combination thereof comprises a ring, a wing, a hook, a clip, a structure comprising teeth, or a combination thereof. In some embodiments, the first extension, said second extension or a combination thereof are positioned so as to be substantially parallel with respect to a long axis of said shunt body. In some embodiments, the first extension, said second extension or a combination thereof may be extended from a position that is substantially parallel to a long axis of said shunt body to one that is at an angle of between about 45 to 120 degrees with respect to a long axis of said shunt body. In some embodiments, the first extension, said second extension or a combination thereof are positioned at an angle of between 45 to 120 degrees with respect to a long axis of said shunt body.

In some embodiments, the shunt body comprises two halves which may be fixedly joined upon insertion through a synovial membrane.

In some embodiments, the shunt comprises at least a first and second extension of the shunt body, which first extension is located proximal to said proximal aperture and which second extension is located proximal to said distal aperture. In accordance with this aspect, the first and second extensions fasten or adjoin the shunt body, such that the shunt is substantially immobilized at a location of placement of the shunt.

In accordance with this aspect, the proximal aperture of the shunt body is positioned proximal to an interior of the joint capsule and the shunt body spans at least a distance substantially equal to that of the synovial membrane, the joint capsule or a combination thereof. In accordance with this aspect, the distal aperture is located substantially outside of at least the synovial membrane.

In one embodiment, the first extension, the second extension or a combination thereof have a length of 2-20 mm. In some embodiments, the side-to-side length of the wings are about 5-30 mm, and in some embodiments, the side-to-side length of the wings are about 1-50 mm, and in some embodiments, the side-to-side length of the wings are about 10-25 mm, and in some embodiments, the side-to-side length of the wings are about 15-35 mm, and in some embodiments, the side-to-side length of the wings is about 20 mm.

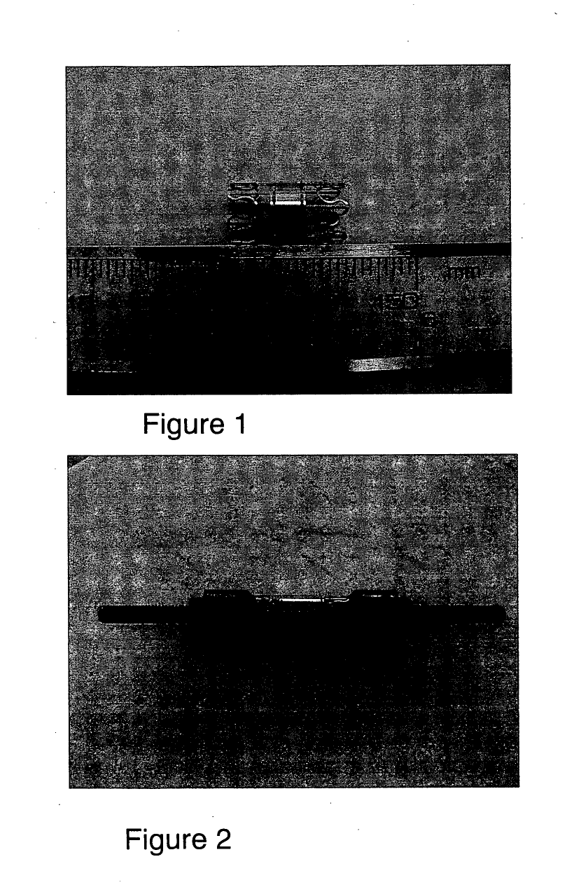

FIG. 1 depicts an embodiment of a shunt of this invention. According to this aspect, the shunt body is of a slightly shorter length (roughly 40 mm) than the proximal and distal extensions in the shunt (roughly 50 mm each). According to this aspect, the shunt contains multiple distal and proximal extensions, which are comparable in number, i.e. the number of distal and number of proximal extensions are equal in this aspect of the invention.

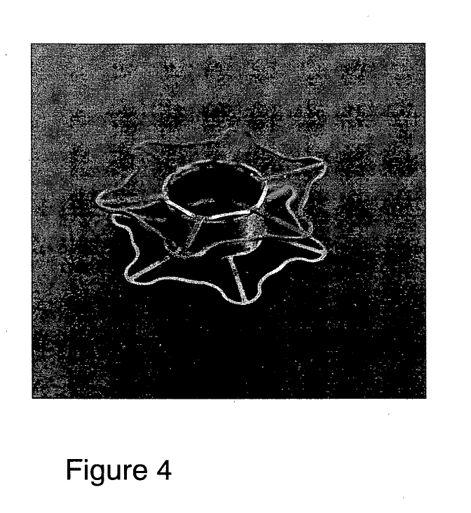

FIG. 4 depicts an embodiment of a shunt of this invention, similar to that shown in FIG. 1. In FIG. 4, the proximal and distal extensions are positioned in their most extended form, such that the long axis of the extension is positioned substantially perpendicularly to the long axis of the shunt body.

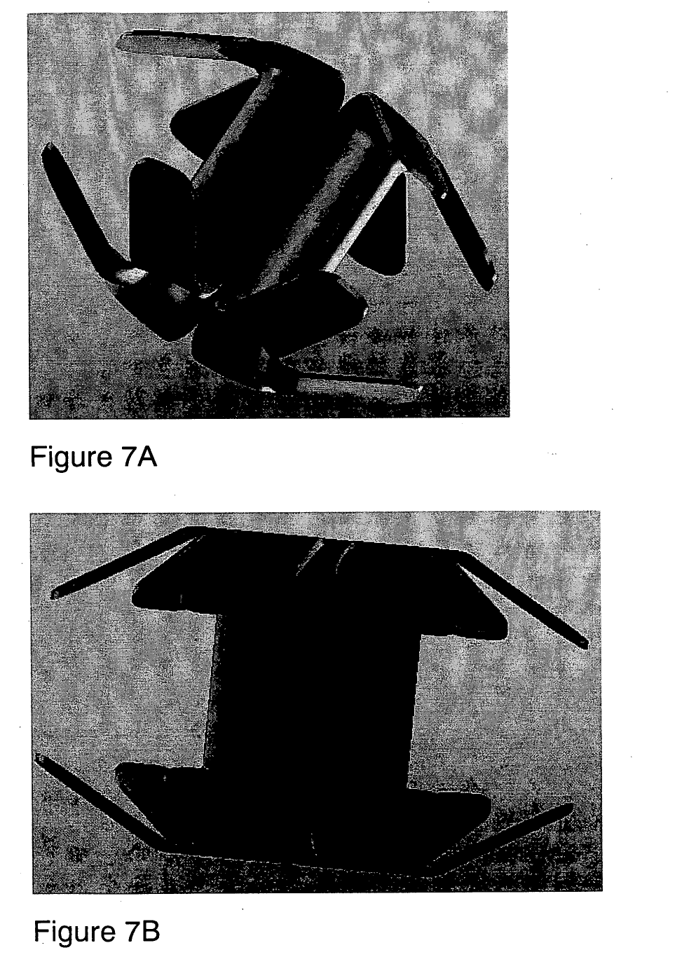

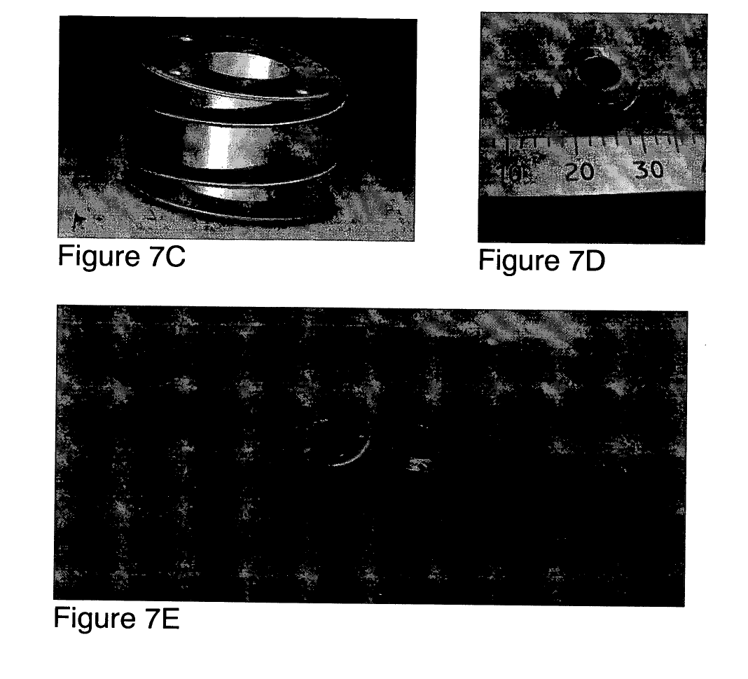

FIG. 7 schematically depicts two views (A and B) of an embodiment of a shunt of this invention, somewhat different from that of FIGS. 1 and 4, in that the extensions are filled in, whereas in the prior figures, such extensions were frame-like and were not filled in. It will be appreciated by the skilled artisan that the extensions may take any shape or form, and be substantially filled in or substantially framed, or some extensions may be substantially filled in and some extensions may be substantially framed, and such choice may reflect the positioning of the shunt within a particular tissue, the depth of the tissue, the condition of the subject into which the shunt is being inserted, or a combination of such considerations, and others, as will be appreciated by the skilled artisan.

FIGS. 7A and 7B further depicts the angling of the extensions such that the long axes of the extensions are approximately 45 degrees with respect to a long axis of the shunt body. In some embodiments, different extensions may be positioned at different angles, with respect to the shunt body long axis, in the same shunt device. FIG. 7C depicts another embodiment of a shunt of this invention, wherein the shunt comprises lateral extensions, angled with respect to a long axis of the shunt body, in addition to the proximal and distal extensions. Such lateral extensions in this aspect may serve to anchor the shunt within a membrane or subcutaneous tissue, acting in some embodiments as a screw, promoting securing of the shunt within the desired position. In some embodiments, such lateral extensions may be of any shape and positioned at any angle with respect to the long axis, such lateral extensions may be coated or treated differentially, or in some embodiments, comparably to the proximal and/or distal extensions or the surface of the shunt body. FIG. 7D depicts the width of an embodiment of a shunt of this invention. FIG. 7E depicts yet another embodiment of a shunt of this invention. According to this aspect, the proximal and distal extensions may be of a different shape, and in some embodiments, may have different widths, geometry or both. It will be appreciated by the skilled artisan that any combination of the features of the shunts shown in the Figures herein may be envisioned, and such figures serve as a guide for the shunts of this invention, but that other shunts may be envisioned based on the description provided herein.

In one embodiment, the term “angle” refers to a measurement of the arc formed by an imaginary line along the long axis of the shunt and an imaginary plumb line perpendicular to the line along the axis of the shunt, with the arc progressing in a clockwise direction around this imaginary plumb line. Thus, in one embodiment, extensions of a shunt of this invention may be positioned at an angle such that the extensions are parallel to the long axis, and therefore the angle would be 0 degrees. In one embodiment extensions of this invention may be positioned parallel to the imaginary plumb line, and therefore the angle would be 90 degrees. In one embodiment, the extension/s is/are positioned at an angle equaling or less than 10 degrees. In one embodiment, the extension/s is/are positioned at an angle equaling or less than 35 degrees. In one embodiment, the extension/s is/are positioned at an angle equaling or less than 55 degrees. In one embodiment, the extension/s is/are positioned at an angle equaling or less than 75 degrees. In one embodiment, the extension/s is/are positioned at an angle equaling or less than 95 degrees. In one embodiment, the extension/s is/are positioned at an angle equaling or less than 115 degrees. In one embodiment, the extension/s is/are positioned at an angle equaling or less than 125 degrees. In one embodiment, the extension/s is/are positioned at an angle of less than 145 degrees. In one embodiment, the extension/s is/are positioned at an angle equaling or less than 165 degrees. In one embodiment, the extension/s is/are positioned at an angle less than 180 degrees.

Thus, it will be apparent to one skilled in the art that the specific positioning of the extensions within a joint capsule affixes the shunt of this invention such that the shunt is most effective for drainage of synovial fluid and/or relief of internal pressure within the joint capsule.

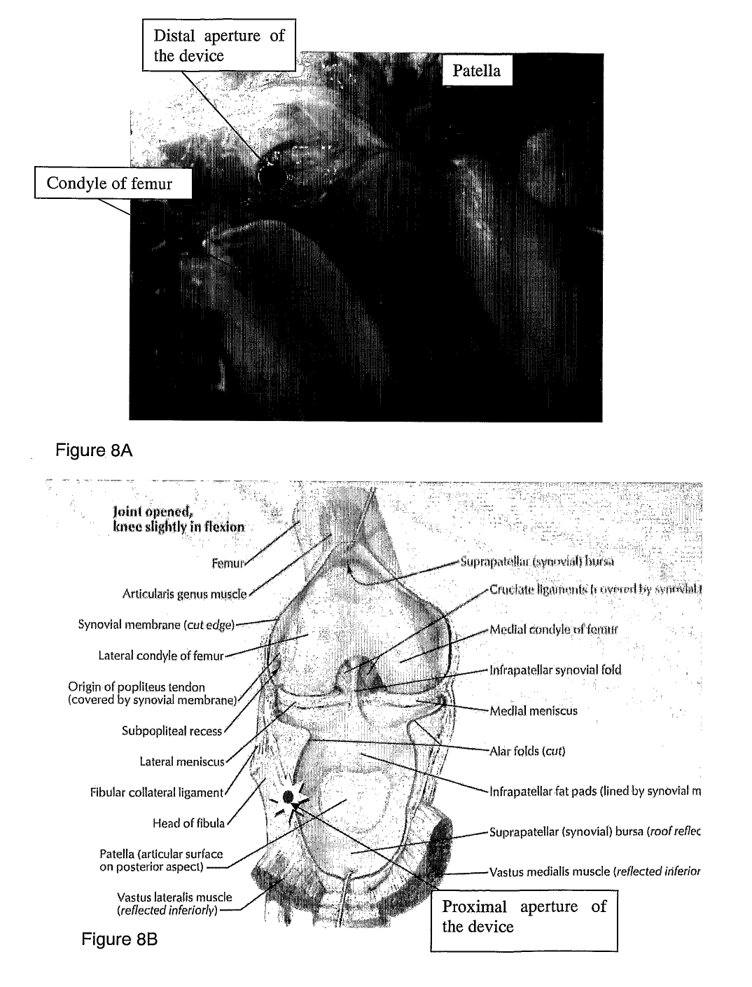

FIGS. 8A and 8B depict implantation of an embodied shunt of this invention within a joint capsule in the knee to facilitate drainage of the fluid from the affected joint. As can be seen in the figure, the positioning of the shunt may be supra-patellar or sub-patellar, at an angle, and so positioned that in some embodiments, such shunt is minimally intrusive upon free mobility about the joint.

In some embodiments, one or more extensions from the shunt body serve to position and confine the shunt at least within the synovial membrane such that the shunt body spans at least the synovial membrane. In one embodiment, the phrase “positions and confines” refers to the capacity of a region to secure a shunt of this invention at a particular location within the indicated site.

In one embodiment of this invention, the shunt is positioned within the synovial membrane and spans at least the length of the snyovial membrane, creating a passageway thereby. In some embodiments, the shunt is positioned at an optimal depth and angle within a site of joint repair, which is at a depth and angle most beneficial for such repair. In one embodiment, the optimal depth and angle that is most beneficial results in positioning the shunt such that a shunt of this invention is accessible to a subcutaneous tissue effective to absorb or remove excess synovial fluid accumulated within such subcutaneous tissue.

This invention provides a therapeutic kit comprising the synovial shunts of this invention. In some embodiments, the kits of this invention further comprise a biocompatible tubing, which tubing is positioned proximal to said distal aperture and which tubing conveys synovial fluid away from an affected joint region. In some embodiments, such tubing serves as a means for active fluid withdrawal, whereby fluid may be withdrawn from the site via extrusion through the tubing. In some embodiments, such tubing serves as a means for passive fluid withdrawal, whereby fluid drains from the site through the tubing and deposits outside the joint capsule, in a subcutaneous region.

In some embodiments, the kits of this invention further comprise a tool for the insertion of said synovial shunt in an affected joint region.

In one embodiment, the present invention provides combined preparations. In one embodiment, the term “a combined preparation” defines especially a “kit of parts” in the sense that the combination partners as defined above can be used independently or in different combinations i.e., simultaneously, concurrently, separately or sequentially.

In some embodiments, the invention provides at least one tool for the insertion of a synovial shunt of this invention.

In some embodiments, the tool is comprised of at least two parts, the first part comprising a more pointed structure, which inserts within a joint capsule. The first part, in some embodiments, is not actually substantially pointed, and in some embodiments, it may terminate in a rounded structure, but it is shaped such that it can insert and potentially pierce the synovial membrane. In some embodiments, such first part is sufficiently shaped to pierce the synovial membrane, however it will not damage other tissue, and sufficient force must be applied to such tool to pierce the membrane, in some embodiments. In some embodiments, such part comprises a locking mechanism, which part prevents a shunt positioned within the tool from dislodging from the tool.

In some embodiments, the tool is comprised of a second part, operationally connected to the first part. In some embodiments, the second part maintains said first extension and said second extension at a position that is substantially parallel to a long axis of said shunt body prior to positioning said shunt in an affected joint. In some embodiments, the operation of said first and second part to insert said shunt into an affected joint region facilitates extension of said first extension and said second extension to be at an angle of between 45 to 120 degrees with respect to a long axis of said shunt body, and such extension is facilitated by a button technology associated with the second part. In some embodiments, the tool specifically contains a safety mechanism, which controls deployment of the extensions, thus ensuring that the shunt does not deploy the extensions within the joint capsule, thereby damaging the joint capsule. In some embodiments, the tool is specifically configured to maintain the shunt therein, ensuring that the shunt is not removed from the tool prior to specific positioning within the capsule, preventing random extrication of the shunt from the tool.

In some embodiments, the tool of this invention is user friendly, and is sized and shaped to optimally insert the shunts within the joint capsule, as herein described. In some embodiments, the tool is so constructed to comprise, for example, a guide which allows for the user to insert the tool at the optimal depth and position for incorporation of the shunt in the desired locale, and in some embodiments, such insertion may be with or without the need for arthroscopy.

In some embodiments, the tool may comprise physical markers and is sized, such that the insertion of the synovial shunts of this invention is optimal in terms of the location and angle of placement within the joint capsule.

In some embodiments, the tool comprises safety controls which regulate deployment of the synovial shunt for implantation. In some embodiments, such controls prevent premature release of the shunt, or optimal placement, which prevents displacement of the shunt upon implantation. In some embodiments, such controls prevent excessive or inappropriate movement of the shunt during positioning of the shunt in implantation. In some embodiments, such controls allow for control during deployment, so that release and deployment of the shunt, in particular, in some embodiments, with regard to deployment of the extensions is specifically controlled.

In some embodiment the tools of this invention are so designed and constructed that manipulation of the synovial shunts within an afflicted capsule is gentle and minimal, and attachment of the shunt therein is with great ease such that little or no necrosis occurs following implantation.

In some embodiments, the shunts and tools as described herein are so constructed and designed such that optimal insertion of the shunt within the capsule is accomplished and maximal elasticity is preserved in the joint.

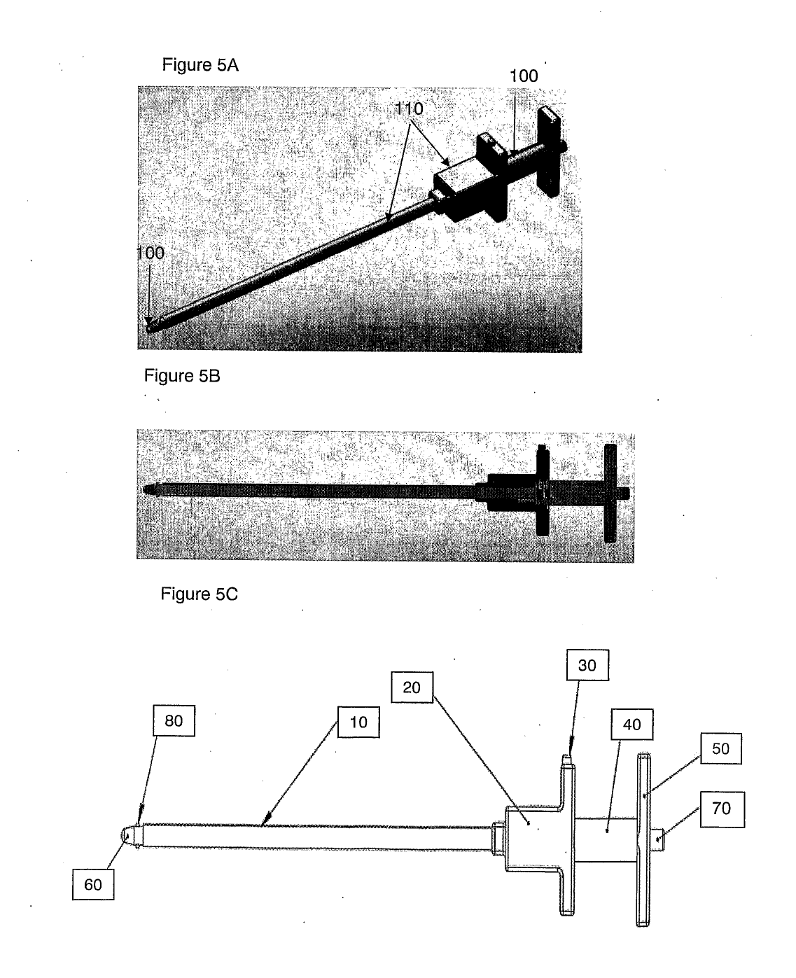

FIG. 5 depicts an embodiment of a tool of this invention. In this aspect, (panel A), the first part (100) culminates in a slightly pointed tip and comprises a push button, which upon depression of the button dislodges the shunt from the device. The shunt is positioned between the first part and the second part in a position such that the extensions are substantially parallel to the long axis of the shunt body (in the non-extended state). The second part of the device (110) contains a push button assembly, for example, which withdraws the second part such that the shunt positioned underneath the second part is partially liberated such that the extensions which were held in an essentially parallel position may now extend. The second part buttons are so configured that a first depression only exposes a first extension, to facilitate individual liberation of the proximally and distally located extensions of the shunt.

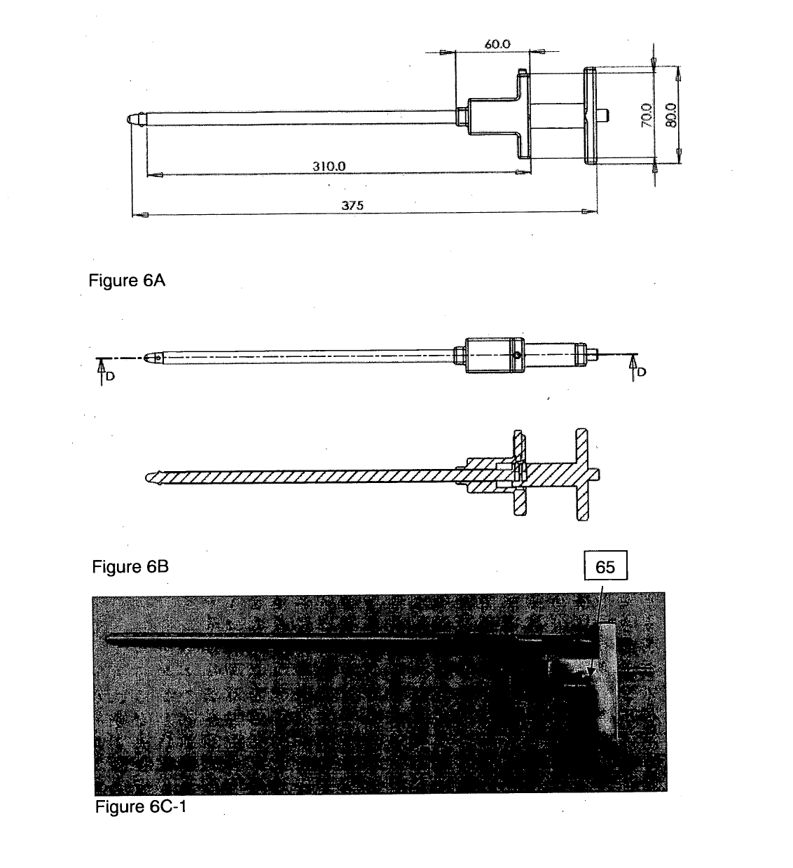

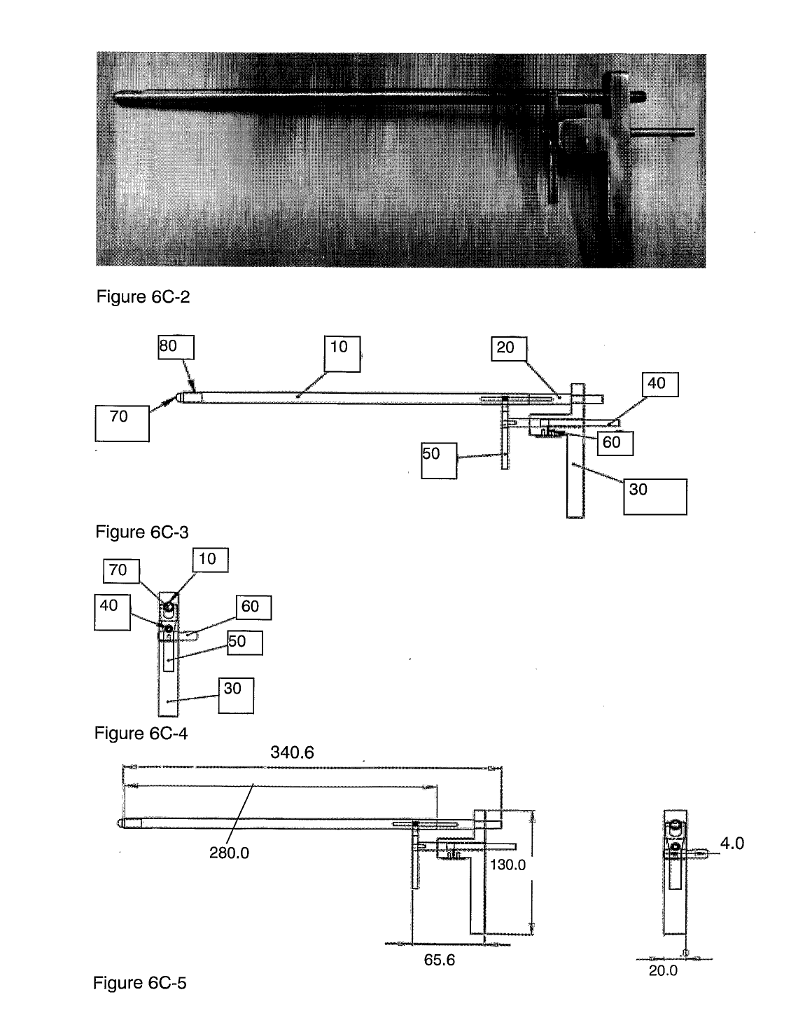

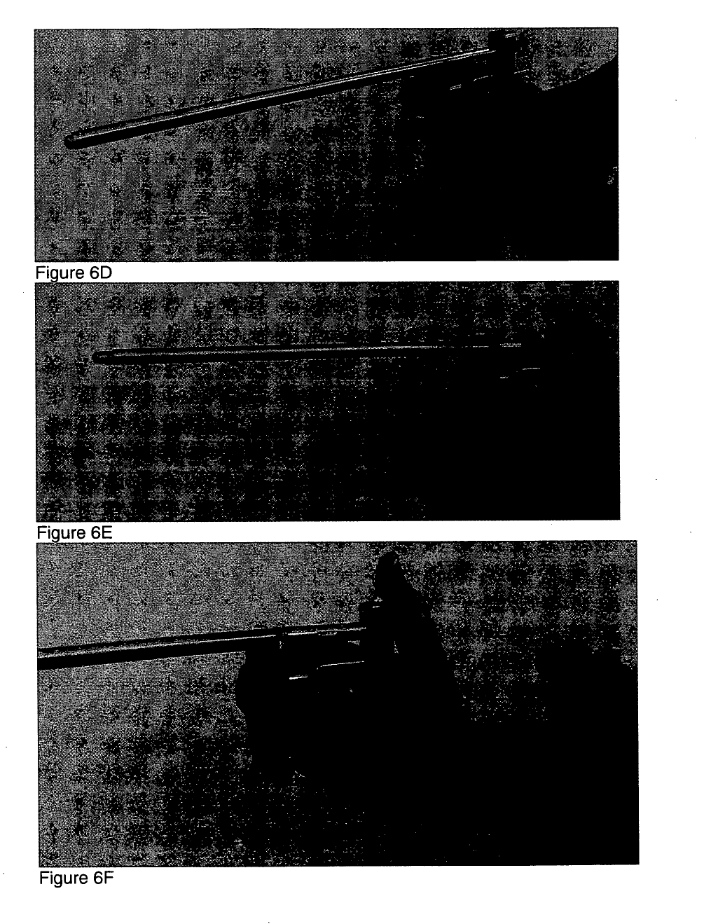

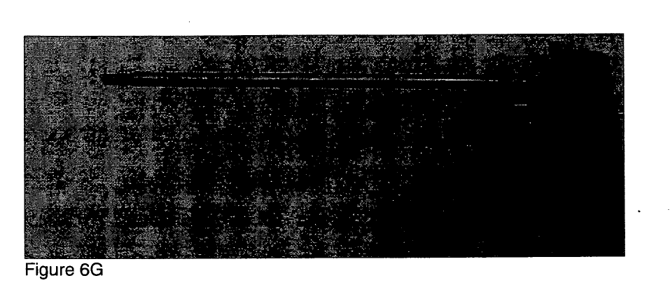

FIG. 6 schematically depicts an embodiment of a tool of this invention. In panel A, an example of dimensions of the tool of this invention is shown and in panel B, an embodiment is depicted, where the push button mechanism is a separate part of the tool.

It is to be understood that many tools may be devised to appropriately insert and position the shunts of this invention, and that the shunt and tools described herein are to be understood to be stand alone devices, which may be used in concert, but this invention is not limited to such combined use.

In one embodiment, this invention provides an instrument to aid in joint repair comprising a tool to guide a shunt of this invention to an optimal angle at a site of repair, a tool to insert a shunt of this invention at a site of joint repair so that the shunt penetrates through the synovial membrane, and inserts there-within, a tool to deploy the shunt extensions to affix the shunt at a desired location, a tool to release a shunt of this invention at a site of repair, or a tool able to provide a combination thereof, whereby the tool may be separated from the shunt following placement of the shunt within a site of joint repair.

In one embodiment, the instrument of this invention comprises at least a single tool. One skilled in the art will recognize that selection of a tool will depend upon the tissue being penetrated.

Preparation of a site of repair may also involve creating a passageway to the site of the joint tissue. Therefore, in one embodiment, a tool of this invention drills a path such to reach the site of repair or reach a site proximal to a site of repair.

In one embodiment, the shunts of this invention separate from the tool following placement of the shunt within the site of repair, and deployment of the extensions such that the shunts of this invention are affixed in their inserted position.

In one embodiment, separation of the tool from the shunt leaves behind the shunt specifically positioned within a site of repair and the mechanism for separation does not cause additional trauma to a site of repair.

In one embodiment, this invention provides a kit for joint treatment comprising the shunt of this invention, at least a tool of this invention, and directions for utilizing the shunt in joint treatment.

One skilled in the art will recognize that choice of a kit by a skilled clinician would be dependent upon factors such as exact nature of the condition being treated, the severity of the condition, the age and general physical condition of the subject, body weight, and response of the individual subject.

Thus, in one embodiment, the kits of this invention contain shunts of different sizes, shapes or chemical compositions, or a combination thereof. In one embodiment, this invention provides a kit for joint repair comprising a shunt of this invention, at least a tool of this invention, and directions for utilizing the shunt in joint repair.

In some embodiments, this invention provides a method for draining synovial fluid from a joint in a subject in need thereof, the method comprising the step of affixing a synovial shunt of this invention in a synovial membrane in an affected joint such that said proximal aperture is positioned proximal to an interior of said joint capsule and said shunt body spans at least a distance substantially equal to that of said synovial membrane, said joint capsule or a combination thereof and said distal aperture is located substantially outside at least said synovial membrane, creating a passageway through said synovial membrane and providing an exit for excess synovial fluid in said joint, thereby being a method for draining synovial fluid from a joint in a subject.

In some embodiments, reference to “affixing” a shunt refers to positioning of the shunt such that its proximal aperture is positioned internal to a joint capsule and its shunt body spans at least across the synovial membrane such that said distal aperture is localized within or past the synovial membrane whereby the shunt body creates a passageway through the synovial membrane to facilitate fluid exit from the joint capsule outside the synovial membrane. The term affixing in this context is to be understood to encompass temporary or permanent insertion as described, via any appropriate means, which enables creation of the passageway.

The passageway serves as a conduit for synovial fluid exit, relief of joint-associated pressure, or a combination thereof.

In some embodiments, this invention provides a method for treating or suppressing, delaying progression or preventing recurrence of a joint disease or disorder in a subject, the method comprising the step of affixing a synovial shunt of this invention in a synovial membrane in an affected joint of said subject, such that said proximal aperture is positioned proximal to an interior of said joint capsule and said shunt body spans at least a distance substantially equal to that of said synovial membrane, said joint capsule or a combination thereof and said distal aperture is located substantially outside at least said synovial membrane, creating a passageway through said synovial membrane and providing an exit for synovial fluid, relief of intra-joint pressure or a combination thereof in said joint, thereby being a method for treating or suppressing, delaying progression or preventing recurrence of a joint disease or disorder in a subject.

In some embodiments, this invention provides for the use of a synovial shunt of this invention in the treatment or suppression, delayed progression or prevention of recurrence of a joint disease or disorder in a subject, wherein said shunt is affixed in a synovial membrane in an affected joint of said subject, such that said proximal aperture is positioned proximal to an interior of said joint capsule and said shunt body spans at least a distance substantially equal to that of said synovial membrane, said joint capsule or a combination thereof and said distal aperture is located substantially outside at least said synovial membrane, creating a passageway through said synovial membrane and providing an exit for synovial fluid, relief of intra joint pressure or a combination thereof in said joint.

In one embodiment, the term “treating” refers to curing a disease. In another embodiment, “treating” refers to preventing a disease. In another embodiment, “treating” refers to reducing the incidence of a disease. In another embodiment, “treating” refers to inducing remission. In another embodiment, “treating” refers to slowing the progression of a disease. The terms “reducing”, “suppressing” and “inhibiting” refer in one embodiment, to lessening or decreasing. The term “progression” may refer to increasing in scope or severity, advancing, growing or becoming worse. The term “recurrence” refers, in one embodiment, to the return of a disease after a remission.

In some embodiments, the affected joint is a knee or hip joint.

In some embodiments, the synovial shunt is positioned at a lateral infra patellar or a lateral supra patellar region.

In some embodiments, the subject is a human subject, or in some embodiments, the subject is an animal subject.

In some embodiments, the subject is afflicted with a full thickness articular cartilage defect; osteoarthritis, infection, rheumatoid arthritis, Gout, reactive arthritis, Bechet's arthritis, Psoriatric arthritis, palindromic arthritis, an autoimmune disease, a joint defect or a defect resulting from trauma, sports, or repetitive stress. In some embodiments, the subject is afflicted with a Baker's cyst or a ganglion cyst.

In some embodiments, this invention provides a method for treating or suppressing, delaying progression or preventing recurrence of a ganglion cyst in a subject, the method comprising the step of affixing a synovial shunt of this invention in a membrane of a ganglion cyst in a subject, such that said proximal aperture is positioned proximal to an interior of said cyst and said shunt body spans at least a distance substantially equal to that of said cyst membrane and said distal aperture is located substantially outside at least said cyst membrane, creating a passageway through said cyst membrane and providing an exit for fluid from within said ganglion cyst, thereby being a method for treating or suppressing, delaying progression or preventing recurrence of a ganglion cyst in a subject.

In one embodiment, the phrase “joint repair” refers to restoring a joint defect to a more healthful state. In one embodiment, restoring a joint, results in regeneration of associated tissue. In one embodiment, joint repair comprises alleviating joint disease (e.g. knee, elbow, hip joints).

In one embodiment, the methods of this invention comprise implanting a shunt of this invention in a subject afflicted with a joint defect or disorder.

In one embodiment, the term “implanting” refers to inserting and fixing a shunt of this invention with in a living site in a subject, the site comprising a site of joint repair and implantation comprising positioning the shunt within the site as herein described.

A clinician skilled in the art will recognize that methods of this invention, which entail implanting a shunt within a site of joint repair, may require preparation of the site of repair. These preparations may occur prior to implantation of the shunt or simultaneously with implantation. For example, fascia and/or other tissues proximal to a site of repair may be cut or moved to reach the site of repair, creating appropriate access for insertion of the shunt used in the methods of this invention. Alternatively, the shunt may be attached to a tool of this invention capable of penetrating through overlying tissues, or a combination thereof. In this case, as the tool penetrates through the overlying tissue to reach the site of repair, the attached shunt is maintained in a manner, which in some embodiments prevents deployment of the extensions.

In some embodiments, implantation of the shunt within a repair site, or several shunts within the repair site, is effected.

In one embodiment, methods of this invention comprise implanting a shunt in a human subject.

In one embodiment, methods of this invention comprise implanting a shunt in a non-human mammalian subject. In one embodiment, methods of this invention comprise implanting a shunt in a horse, a race horse, a cow, a steer, a pig, a sheep, a farm animal, a pet, such as a dog, or a cat.

In one embodiment, methods of this invention are utilized for induced or enhanced repair of a joint defect or disorder. In one embodiment, the joint defect results from a trauma, a sports injury, a full thickness articular cartilage defect, a joint defect, a repetitive stresses injury (e.g., osteochondral fracture, secondary damage due to cruciate ligament injury) and others, as will be appreciated by the skilled artisan.

In one embodiment, the joint disorder comprises a disease of the cartilage. In one embodiment, methods of this invention induce or enhance joint repair in osteoarthritis, rheumatoid arthritis, aseptic necrosis, osteochondritis dissecans, articular cartilage injuries, chondromalacia patella, chondrosarcoma, chondrosarcoma—head and neck, costochondritis, enchondroma, hallux rigidus, hip labral tear, osteochondritis dissecans, torn meniscus, relapsing polychondritis, canine arthritis, fourth branchial arch defect and others.

In one embodiment, methods of this invention induce or enhance joint repair in degenerative cartilagenous disorders comprising disorders characterized, at least in part, by degeneration or metabolic derangement of connective tissues of the body, including the joints or related structures, tendons, and fibrous tissue.

In one embodiment, a joint defect or disorder repaired by the methods of this invention utilizing any shunt and/or at least a tool of this invention.

In one embodiment, the 3-D shape and chemical composition of a shunt of this invention, used in the methods and/or kits of this invention will be determined by skilled clinicians, based on factors such as exact nature of the condition being treated, the severity of the condition, the age and general physical condition of the subject, body weight, subcutaneous tissue thickness and response of the individual subject, etc.

In some embodiments, the implantation of the shunts of this invention within a joint capsule are at a region in the capsule, as herein described, which region results in no diminished motion capacity for the subject, as the insertion does not compromise motion or minimally compromises motion.

In some embodiments, such positioning ensures that the shunt is not placed on a muscle, which can block proper drainage through the shunt.

In some embodiments, the implantation within the capsule is optimal for preserving the strength and elasticity of the capsule.

In one embodiment, the specific positioning of a shunt of this invention during methods of this invention will be determined by skilled clinicians, based on factors such as exact nature of the condition being treated, the severity of the condition, the age and general physical condition of the subject, body weight, and response of the individual subject, etc.

In one embodiment, methods of this invention are evaluated by examining the site of joint tissue repair, wherein assessment is by histology, palpation, endoscopy, arthroscopy, or imaging techniques comprising X-ray photographs, computerized X-ray densitometry, computerized fluorescence densitometry, magnetic resonance imaging or another method known in the art, or any combination thereof. Such methods will attest to the improvement of the joint treated, lack of deleterious effects of conveying fluid to subcutaneous sites, for example, and in some embodiments, via demonstration of no pain or exaggerated swelling at the subcutaneous site to which fluid is conveyed. The skilled artisan will appreciate that standard methodology will be utilized to assess the improvement effected by implantation of the shunts of this invention.

In some embodiments, the term “comprise” or grammatical forms thereof, refers to the inclusion of the indicated components of this invention, as well as inclusion of other active agents, and pharmaceutically acceptable carriers, excipients, emollients, stabilizers, etc., as are known in the pharmaceutical industry.

### EXAMPLES

### Example 1

### Preparation of an Embodiment of a Joint Shunt of this Invention

A nitinol tube was prepared by standard methodology having an inner diameter (ID) of 8.98 mm, an outer diameter (OD) of 10 mm, which was subsequently ground to an OD of 9.48 mm. The tube was cut to desired lengths, for example, a 13.5 mm tube length was cut for a shunt having a 3.5 mm lumen, and a 16 mm tube length was cut for a device having a 6 mm lumen using a laser cutting tool. Termini of the tube were further cut to form extensions, which may be bent at desired angles following heat treatment, as described further hereinbelow. FIG. 1 depicts an example of a cut nitinol tube prior to angling of the terminal extensions of the tube.

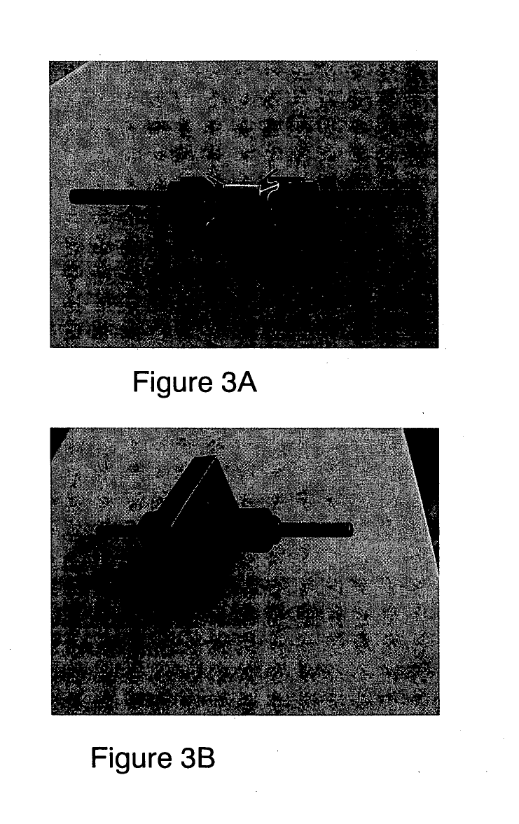

The cut nitinol tubes were then heat treated. The cut nitinol tubes were loaded on appropriate jigs (FIG. 2) containing a shaft having a diameter which is slightly smaller than that of the cut nitinol tube, and terminally placed nuts, which may be advanced toward each other along the length of the shaft. Angled extensions were created by advancing the nuts toward each other along the length of the shaft, thereby exerting pressure on the wings and positioning them to their desired position (FIGS. 3A and 3B).

The jig containing the loaded device was placed in a 630° C. oven for 10 minutes, after which the jig and device were cooled in cold water and the device was ejected from the jig (FIG. 4).

### Example 2

### Implantation of an Embodiment of a Joint Shunt of this Invention in a Joint

The desired implantation location in the lateral supra patella was marked in a goat, at 1 cm above the patella at a 45° angle lateral to the knee. An incision to the knee capsule was made and the skin and fascia were separated. Tissue thickness was measured with a caliper. An embodiment of the delivery system was inserted into the knee capsule, maintaining the shunt in its unextended form prior to insertion. Once the delivery tool inserted within the knee capsule, the handle of the device was rotated, which in turn facilitated extension of the proximal extensions in the device. The delivery system was withdrawn to its desired length and location and the second or distal extensions were then extended, constraining the device in its desired location. In this example, the distal nitinol “wings” were pressed against the inner membrane and the capsule wall. The delivery system was then removed from the subject and the skin was stitched closed.

### Example 3

### An Embodiment of a Joint Shunt Delivery System of this Invention

FIG. 5 depicts multiple views of an embodiment of a delivery system of this invention. The delivery system provides control over each stage of the device deployment in order to reduce the risk of open the entire device inside the knee capsule.

The device contains an overtube (10) operationally connected to an overtube handle (20) containing a button (30), which regulates deployment of the device, which is held fixed on the dilator edge (60) along the dilator (40) positioned inside the overtube by the locking mechanism (80), controlled by the locking button (70) (FIG. 5C).

The movement of the over tube (10) is controlled in some embodiments by a double press button mechanism (30), each press on the button allows for deployment of one the two extensions of the shunt devices of this invention. A first press results in a backward pull on the over tube of about 6 mm thereby deploying the proximal wings of the implant. A second press results in another backward pull on the over tube of about 6 mm, thereby deploying the distal wings.

The overtube is positioned over the dilator (40), which is a long ‘ball lock pin’, locking the implant on the dilator during the deployment process using locking balls (80) positioned near the dilator edge (60). When the implant is fully deployed and fixed in place, then the locking button (70) is depressed the implant is released from the dilator.

A dimensional and section view of this embodiment of a delivery system is depicted in FIGS. 6A and 6B, respectively. FIG. 6A for example provides approximate dimensions for an embodiment of a tool for use in deploying the embodied devices exemplified herein. It is to be understood that tool dimensions may be adjusted to accommodate changes in size, shape or configuration of the shunts of this invention. In some embodiments, a stopper mechanism is incorporated in the tools of this invention, which in some embodiments, operates via engagement of spring pins, as can be seen in FIG. 6B.

FIGS. 6C-1-5 depict another embodiment of a tool demonstrating certain safety features incorporated in the tools of this invention, which are important for safe positioning and deployment of the shunts of this invention. FIGS. 6C 1-5 depict an embodied tool of this invention, and highlights features of the tool. The tool comprises an over tube (10), a dilator (20), a pistol grip (30), a trigger pin (40), a trigger (50), a trigger stopper (60), a trigger stopper safety catch (65), a dilator edge (70), and a demarcation point for localization of the shunt (80). The delivery systems/tools of this invention allow for the controlled positioning and deployment of the shunts of this invention. Such control allows for inter alia, the safe deployment of such shunts, preventing or minimizing the risk of deployment of the shunt within the capsule, prior to appropriate deployment and positioning therein.

FIG. 6C-5 depicts certain embodiments of the dimensions of certain elements of the tools of this invention, according to this aspect.

FIG. 6D-6G depict an embodiment of how the tool may be utilized to position and deploy the shunts of this invention. The movement of the over tube (10) is controlled by the trigger (50) and trigger stopper mechanism (60) (FIGS. 6D and 6E). Each press on the trigger stopper (50) allows for one step of the shunt deployment. The first press on the trigger stopper (60) allows for the pulling of the trigger (50) (and the over tube (10)) 6 mm backward to deploy the proximal wings of the implant. A second press on the trigger stopper (60) may be accomplished only after removing the trigger stopper safety catch (65), which adds an additional control measure preventing random deployment of both proximal and distal extensions. The second press allows for the pulling of the trigger (and the over tube (10)) until the distal wings deploy (FIGS. 6F and 6G). In some embodiments, the dilator (20) may comprise a long ‘ball lock pin’ mechanism, locking the implant on the dilator during the deployment process using locking balls on the distal side of the tube, close to the dilator edge (70), and only when the implant is fully deployed and fixed in his place, is the ball lock pin mechanism button pushed and shunt release from the dilator is accomplished.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1-49. (canceled)

50. A synovial shunt for insertion in a body joint, which shunt creates a passageway between an interior of a joint capsule and an exterior of said joint capsule, said shunt consisting of:
a shunt body comprised of a biocompatible material, which is substantially hollow;
a proximal and distal aperture flanking said shunt body; and
at least a first and second extension of said shunt body, which first extension is located proximal to said proximal aperture and which second extension is located proximal to said distal aperture, wherein said first and second extensions fasten or adjoin said shunt body, such that said shunt is substantially immobilized at a location of placement of said shunt;
wherein
said proximal aperture of said shunt body is positioned proximal to an interior of said joint capsule and said shunt body spans at least a distance substantially equal to that of said synovial membrane, said joint capsule or a combination thereof and said distal aperture is located substantially outside of at least said synovial membrane; and
the ratio between said shunt body diameter and said shunt body length is greater than 0.5.

51. The synovial shunt of claim 50, wherein said distal aperture is located proximal to a subcutaneous tissue and said shunt body spans a length across a synovial membrane and extending into a subcutaneous tissue,
said subcutaneous tissue is a muscle, a vein, fat, a ligament or a tendon, and
said distal aperture is located near an exterior of a joint capsule.

52. The synovial shunt of claim 50, wherein said shunt body has a thickness of between 50-300 μm,
said shunt body has a diameter of between 1-25 mm, and
said shunt body has a length of between 3-10 mm.

53. The synovial shunt of claim 50, wherein said shunt body length varies as a function of the thickness of subcutaneous tissue into which said synovial shunt will be implanted.

54. The synovial shunt of claim 50, wherein said first extension, said second extension or a combination thereof have a length of 2-20 mm.

55. The synovial shunt of claim 50, wherein said shunt is comprised of a metal, a ceramic, or a polymer.

56. The synovial shunt of claim 55, wherein said shunt is prepared from a single piece of metal.

57. The synovial shunt of claim 55, wherein said metal is nitinol, stainless steel, or titanium.

58. The synovial shunt of claim 55, wherein said polymer comprises a natural polymer comprising, collagen, elastin, silk, hyaluronic acid, chytosan, or any combinations thereof.

59. The synovial shunt of claim 55, wherein said polymer comprises a synthetic biodegradable polymer.

60. The synovial shunt of claim 59, wherein said synthetic biodegradable polymer comprises alpha-hydroxy acids including poly-lactic acid, polyglycolic acid, enantioners thereof, co-polymers thereof, polyorthoesters, or combinations thereof.

61. The synovial shunt of claim 50, wherein a luminal surface of said shunt, an exterior surface of said shunt or a combination thereof is treated to reduce adhesion of cells or particulate matter thereto.

62. The synovial shunt of claim 61, wherein said shunt comprises a coating, which diminishes or abrogates adhesion thereto.

63. The synovial shunt of claim 61, wherein said shunt comprises a positively charged material or incorporates a positively charged material.

64. The synovial shunt of claim 61, wherein said first extension, said second extension or a combination thereof are treated to reduce adhesion of cells or particulate matter thereto.

65. The synovial shunt of claim 61, and wherein said first extension, said second extension or a combination thereof are treated to promote adhesion to cells in a region to which said shunt is adhered.

66. The synovial shunt of claim 50, wherein a luminal surface of said shunt, an exterior surface of said shunt or a combination thereof comprises a therapeutic agent,
wherein said therapeutic agent comprises a growth factor, an agent which aides in wound repair, or a combination thereof and
wherein said therapeutic agent comprises an anticoagulant, an anti-inflammatory compound, or a combination thereof.

67. The synovial shunt of claim 50, wherein said first extension, said second extension or a combination thereof comprises a ring, a wing, a hook, a clip, a structure comprising teeth, or a combination thereof.

68. The synovial shunt of claim 50, wherein said first extension, said second extension or a combination thereof are positioned so as to be substantially parallel with respect to a long axis of said shunt body and
wherein said first extension, said second extension or a combination thereof may be extended from a position that is substantially parallel to a long axis of said shunt body to one that is at an angle of between 45 to 120 degrees with respect to a long axis of said shunt body.

69. The synovial shunt of claim 50, wherein said first extension, said second extension or a combination thereof are positioned at an angle of between 45 to 120 degrees with respect to a long axis of said shunt body.

70. The synovial shunt of claim 50, wherein said shunt body comprises two halves which may be fixedly joined upon insertion through a synovial membrane.

71. A therapeutic kit comprising the synovial shunt of claim 50, optionally further comprising a biocompatible tubing, which tubing is positioned proximal to said distal aperture and which tubing conveys synovial fluid away from an affected joint region, optionally further comprising a tool for the insertion of said synovial shunt in an affected joint region
wherein said tool is comprised of at least two parts, said first part comprising a pointed structure, which inserts within a joint capsule and said second part, operationally connected thereto, which second part delivers said synovial shunt to said joint capsule such that a proximal aperture of said shunt body is positioned proximal to an interior of said joint capsule
wherein said second part maintains said first extension and said second extension at a position that is substantially parallel to a long axis of said shunt body prior to positioning said shunt in an affected joint, and
wherein operation of said first and second part to insert said shunt into an affected joint region facilitates extension of said first extension and said second extension to be at an angle of between 45 to 120 degrees with respect to a long axis of said shunt body.

72. A method for draining synovial fluid from a joint in a subject in need thereof, the method comprising the step of affixing the synovial shunt of claim 50 in a synovial membrane in an affected joint such that said proximal aperture is positioned proximal to an interior of said joint capsule and said shunt body spans at least a distance substantially equal to that of said synovial membrane, said joint capsule or a combination thereof and said distal aperture is located substantially outside at least said synovial membrane, creating a passageway through said synovial membrane and providing an exit for excess synovial fluid in said joint, thereby being a method for draining synovial fluid from a joint in a subject
wherein said affected joint is a knee or hip joint,
wherein said synovial shunt is positioned at a lateral infra patellar or a lateral supra patellar region,
wherein said subject is a human subject, or said subject is an animal subject, and
wherein said subject is afflicted with a full thickness articular cartilage defect; osteoarthritis, infection, rheumatoid arthritis, an autoimmune disease, a joint defect or a defect resulting from trauma, sports, or repetitive stress or
wherein said subject is afflicted with a Baker's cyst.

73. A method for treating a joint disease or disorder in a subject, the method comprising the step of affixing the synovial shunt of claim 50 in a synovial membrane in an affected joint of said subject, such that said proximal aperture is positioned proximal to an interior of said joint capsule and said shunt body spans at least a distance substantially equal to that of said synovial membrane, said joint capsule or a combination thereof and said distal aperture is located substantially outside at least said synovial membrane, creating a passageway through said synovial membrane and providing an exit for synovial fluid, relief of intra joint pressure or a combination thereof in said joint, thereby being a method for treating a joint disease or disorder in a subject,
wherein said affected joint is a knee or hip joint,
wherein said synovial shunt is positioned at a lateral infra patellar or a lateral supra patellar region,
wherein said subject is a human subject or
wherein said subject is an animal subject,
wherein said subject is afflicted with a full thickness articular cartilage defect; osteoarthritis, rheumatoid arthritis, joint inflammation, infection, a joint defect or a defect resulting from trauma, sports, or repetitive stress.

74. A method for treating a ganglion cyst in a subject, the method comprising the step of affixing the synovial shunt of claim 50 in a membrane of a ganglion cyst in a subject, such that said proximal aperture is positioned proximal to an interior of said cyst and said shunt body spans at least a distance substantially equal to that of said cyst membrane and said distal aperture is located substantially outside at least said cyst membrane, creating a passageway through said cyst membrane and providing an exit for fluid from within said ganglion cyst, thereby being a method for treating a ganglion cyst in a subject.
